SEQUENCE LISTING (1 sequence)

SEQ ID NO: 1 - protein, 146 aa, Syrian Hamster
GGGWGQGGGTHNQWNKPSKPKTNMKHMAGAAAAGAVVGGLGGYMLGSAMSRPMMHFGNDWEDRYYRENMNRYPNQVYYRPVDQYNNQNNFVHDCVNITIKQHTVTTTTKGENFTETDIKIMERVVEQMCTTQYQKESQAYYDGRRS

(19) United States
(12) Patent Application Publication
(10) Pub. No.: US 2006/0110785 A1
(43) Pub. Date: May 25, 2006

(54) Methods to differentiate protein conformers

(75) Inventors: Bruce C. Onisko, Kensington, CA (US); Christopher J. Silva, Albany, CA (US); Jesus R. Requena, Briom (ES)

Correspondence Address: USDA-ARS-OFFICE OF TECHNOLOGY TRANSFER;PATENT ADVISORS OFFICE, WESTERN REGIONAL RESEARCH CENTER, 800 BUCHANAN ST, ALBANY, CA, 94710, US

(73) Assignees: The U.S.A. as rep. by the Secretary of Agriculture, Washington, DC (US); Universidade De Santiago De Compostela, Santiago de Compostela (ES)

(21) Appl. No.: 11/249,216

(22) Filed: Oct. 13, 2005

Related U.S. Application Data
(60) Provisional application No. 60/618,952, filed on Oct. 15, 2004.

(51) Int. Cl. C12Q 1/37; G06F 19/00

(52) U.S. Cl. 435/23; 702/19

(57) ABSTRACT

The invention is directed to methods to distinguish among different protein conformers of the same protein such as proteins which form amyloid deposits. Using the methods of the invention, one or more protein conformers in a sample can be detected, differentiated, and quantitated. An example of a protein which is known to exist in at least two conformations is the normal prion protein (PrP^{C}) and its infectious isoform (PrP^{Sc}). The invention provides means to distinguish PrP^{C} from PrP^{Sc} and allows quantitation of each individually, even when the conformers are present together in a mixture. Thus, the methods of the invention can provide important tools for human and animal health.

### BACKGROUND OF THE INVENTION

1. Field of the Invention

The present invention relates to methods for detecting one or more protein conformers in a sample containing a protein having at least two protein conformations. The invention is also directed to methods for distinguishing and quantitating stable protein conformations of the same protein. An example of a protein which is known to exist in at least two conformations is the normal prion protein (PrP^{C}) and its infectious isoform (PrP^{Sc}).

2. Description of the Art

The vast majority of proteins adopt only one stable conformation. Exceptions exist, and many of these alternately folded proteins polymerize to form amyloid deposits with serious consequence to health. The amyloid deposits, that is, abnormal deposits of aggregated protein, are found either in cells or in the space between cells and are usually resistant to proteolysis. At least sixteen types of human disease are associated with fibrils made of abnormally folded proteins (Pepys (1996)). These include spongiform encephalopathies, Alzheimer's disease, Parkinson's disease, Huntington's disease, and type II diabetes.

An example of a protein which causes disease when misfolded is the prion protein. Prions, such as mammalian PrP and fungal sup35, are unique amongst amyloidogenic proteins in that they are known to exist in more than two stable conformations. Prion diseases have properties that are maintained upon transmission from one host to the next, allowing different prion “strains” to be distinguished. The strains cause specific phenotypes, such as different symptomology (ataxias, hyperactivity, lethargy), time from exposure to disease, and different tissue distribution of PrP^{Sc}.

A critical difference between prions and other amyloids is that prions are by definition infectious (Prusiner (1982)). Very substantial and diverse evidence suggests that transmissible spongiform encephalopathies (TSEs), a group of fatal neurodegenerative diseases affecting humans and animals, are mediated by a prion, named PrP (prion protein) ((Prusiner (1982); Prusiner (1998); Soto and Castilla (2004); Aguzzi and Polymenidou (2004); and Prusiner (1991)). The most widely studied TSEs in food-producing animals include scrapie in sheep and goats, bovine spongiform encephalopathy (BSE) in cattle, and chronic wasting disease (CWD) in mule deer and elk. Other TSEs in animals included transmissible mink encephalopathy (TME) in mink and feline spongiform encephalopathy (FSE) of cats. Prion diseases of humans have also been identified. These include: Creutzfeldt-Jakob Disease (CJD); Gerstmann-Straussler-Scheinker Syndrome (GSS); Fatal Familial Insomnia (FFI), and Kuru.

PrP exists in at least two conformations, PrP^{C} and PrP^{Sc}. The latter is associated with TSEs, and PrP showing the physico-chemical characteristics of PrP^{Sc} is isolated as the main and most probably only component of the TSE infectious agent. PrP^{C} can be converted into PrP^{Sc}, in the presence of pre-formed PrP^{Sc}, through a poorly understood molecular process (Aguzzi and Polymenidou (2004); Prusiner (1991); and Come et al. (1993)). The structure of PrP^{C} has been characterized by NMR (Riek et al. (1996)), but that of PrP^{Sc} is largely unknown, as its insolubility in non-denaturing solvents has seriously hampered analytical efforts. It is known, however, that PrP^{Sc} and PrP^{C} differ with respect to secondary, tertiary and quaternary structures (Prusiner (1998)). No covalent differences have been detected between the two molecules (Stahl et al. (1993)), although the possibility that post-translational modifications of a small set of PrP^{C} molecules could trigger structural changes relevant to initiation of conversion to PrP^{Sc} cannot be ruled out (Requena et al. (2001)). Studies using Fourier transform infrared spectroscopy (FTIR) indicate that PrP^{Sc} contains an increased fraction of β-sheet and decreased fractions of a-helix and random coil with respect to PrP^{C} (Prusiner (1998)). PrP^{C} is a monomeric protein anchored to the cell membrane through a glycan phosphoinositol (GPI) anchor. In contrast, PrP^{Sc} is isolated as an aggregate. PrP^{Sc} is partially resistant to proteinase K (PK), that trims an amino terminal segment of the protein generating a well defined resistant core termed PrP 27-30 whereas PrP^{C} is rapidly degraded by PK (Prusiner (1998)). PrP 27-30 retains the infectious character, and hence the essential structural characteristics, of PrP^{Sc}, with the trimmed aminoterminal domain probably consisting of a highly flexible tail as seen in PrP^{C}. In the presence of detergent, PrP 27-30 further polymerizes to rod-shaped filaments with the tinctorial properties of amyloid (McKinley et al. (1991)).

At present, protein conformers can be discriminated by methods such as (FTIR) or circular dichroism (CD) spectroscopy, but only when the proteins have been extensively purified. NMR and X-ray diffraction, the most common methods to determine protein structure, have been used successfully to determine the three dimensional structure of the soluble forms of amyloidogenic proteins (e.g., the cellular form of the prion protein PrP^{C}), however these methods can not be used for the amyloids themselves since amyloids by nature are neither soluble, as required by NMR, nor crystallizable, as required by high resolution X-ray diffraction. Considerable effort has gone towards attempts to find antibodies that discriminate PrP^{Sc} from PrP^{C} but to date, no antibodies have been found that selectively bind PrP^{Sc}.

In view of the considerable human and animal health considerations related to alternately folded proteins that form amyloid deposits, what is needed are methods to detect, distinguish, and, if desired, quantitate two or more protein conformations of the same protein.

### SUMMARY OF THE INVENTION

The invention comprises methods to distinguish among different protein conformers of the same protein such as proteins which form amyloid deposits. Using the methods of the invention, one or more protein conformers in a sample can be detected, differentiated, and quantitated. Thus, for example, in the case of prion protein conformers, the method provides a means to distinguish PrP^{C} from PrP^{Sc} and allows quantitation of each individually, even when the conformers are present together in a mixture. Because the methods of the invention distinguish among conformers, samples having only one protein conformer can be identified. This is useful to identify the presence of amyloid diseases in humans or animals. Thus, the methods of the invention can provide important tools for human and animal health.

The method of the invention for detecting, distinguishing or quantitating a protein conformer in a sample comprising a protein having a plurality of protein conformers, wherein each conformer is characterized by a unique protein conformation, comprises:
(a) reacting the sample with a protein-modifying reagent which reacts differentially with each of the plurality of protein conformers under conditions whereby the reagent forms one or more covalent bonds with a first one of the plurality of protein conformers to form a first unique entity, and a second entity corresponding to each additional protein conformer results either because the reagent does not form a covalent bond or bonds with a second one of the plurality of protein conformers or because the reagent forms a covalent bond or bonds with a second one of the plurality of protein conformers wherein the covalent bond or bonds is or are different from the covalent bond or bonds formed with the first protein conformer;
(b) treating the reacted sample of step (a) with a protein-cleaving reagent under conditions whereby a peptide bond or bonds in the first entity is cleaved to form at least one unique modified peptide and whereby a peptide bond or bonds in the reacted second entity is cleaved to form a unique different peptide which differs from the unique modified peptide of the first entity; and
(c) analyzing the treated sample of step (b) to determine the presence of the unique modified peptide of the first entity or to determine the presence of the unique different peptide of the second entity.

The invention can also include the steps of analyzing for both the unique modified peptide of the first entity and the presence of the unique different peptide of the second entity. The invention methods further encompass the embodiment wherein the plurality of protein conformers includes more than two and wherein one or more of the protein conformers is quantitated. The peptides may be identified by various methods such as: mass spectrometric, calorimetric, immunometric, fluorometric, or radiometric detectors with or without prior chromatographic separation.

In sum, the invention provides a means for distinguishing among protein conformers. Using the methods of the invention, chemical markers can be created that are unique to each conformer of the same protein, and the ratio or absolute amounts of each conformer can be determined.

The invention fulfills an important need of providing means to detect and distinguish different protein conformers of a protein such as one which forms amyloid deposits.

An example of a protein which is known to exist in at least two conformations is the normal prion protein (PrP^{C}) and its infectious isoform (PrP^{Sc}). PrP^{Sc} is found in the brain of mammals infected with TSE diseases, such as CJD, BSE, CWD, TME, and scrapie. These conformers have different biological properties and are important to human health and animal health. They can be transmitted from person to person, animal to animal, and animal to people. Currently, there is no means of identifying asymptomatic carriers or infected, but asymptomatic people or animals. Thus, the ability to identify such persons or animals would be a benefit to human and animal health.

The invention provides an important public safety tool. There is considerable interest in methods to demonstrate the safety of beef and beef products worldwide. The methods of the invention fulfill the need of providing an assay to detect low levels of prions in live animals.

It is an object of the invention to enable the differentiation of proteins like PrP^{Sc} that are known to exist in more than two conformers, colloquially known as “strains”. The implications to agriculture of the different prion strains may be enormous. For example, the invention could be used to determine the etiology of TSE outbreaks or determine the genesis of new TSE strains.

Conformational difference may be a form of protein regulation, so distinguishing conformations may be used to identify conformers that regulate cellular processes. For example, cytosolic polyadenylation element binding protein exists in at least two conformations which are believed to have different biochemical properties. Distinguishing between conformations would allow the testing of this hypothesis, and further elucidate the mechanism of long term potentiation in neurons.

The differing reactivity of amino acid residues would allow the relative position of amino acids to be determined, thus giving structural information that is not obtainable from any other means and providing key information to assist modeling of protein structures that can not be studied by NMR spectrometry or X-ray diffraction.

Other objects and advantages of the invention will become readily apparent from the ensuing description.

### BRIEF DESCRIPTION OF THE DRAWINGS

FIG. 1 shows cross-linking of PrP27-30 by BS³. As discussed in detail in the Materials and Methods of Example 1, below, PrP27-30 was treated with 1.7 mM BS³ at 37° C. for 30 minutes. The reaction was quenched with lysine and PrP 27-30 submitted to SDS-PAGE with or without previous deglycosylation with PNGase F. The gel was stained with Coomassie blue.

FIG. 2 shows mass spectra of cross-linked monomer and dimer and of control deglycosylated PrP27-30. Bands were excised from gels and digested in gel with trypsin overnight at 37° C. Peptides were extracted, dried in vacuo and analyzed by matrix assisted laser desorption ionization-time of flight (MALDI-TOF) as described. (A) Spectra of control, monomer and dimer digests. The main identified peaks are indicated. For a more comprehensive list of peaks see Table 2. (B) Expanded area showing decrease of the G₉₀-K₁₀₆ aminoterminal peptide.

FIG. 3 shows comparison of the relative intensities of amino terminal tryptic peptides from PrP 27-30 identified by MALDI-TOF or nanoLC-ESI-Q-TOF mass spectrometry.

FIG. 4 shows detection of cross-linked peptides in trypsin digests of BS³-cross-linked PrP 27-30 by nanoLC/MS.

FIG. 5 shows confirmation of an intermolecular cross-link involving two G₉₀ amino termini in cross-linked PrP27-30 by MS/MS.

FIG. 6 shows detection of a cross-linked peptide, G₉₀-K₁₀₁—X-G₉₀-K₁₀₁, in the lysC digest of BS³-cross-linked PrP 27-30 by MALDI-TOF. Bands were excised from gels and digested in gel with lysC overnight at 37° C. Peptides were extracted, dried in vacuo and analyzed by MALDI-TOF as described in Example 1, below.

### DETAILED DESCRIPTION OF THE INVENTION

In its broadest ambit, the invention is directed to methods to distinguish different stable conformations of the same protein. The invention is also directed to methods wherein the distinguished conformations are also quantitated. Thus, using the methods of the invention, two or more different conformations of the same protein such as one which forms amyloid deposits can be distinguished, and each one can be quantitated individually, even when present in a mixture.

While the methods of the invention are useful to distinguish among a plurality of protein conformers of the same protein, the invention is first described wherein the protein is one which exists as two different conformations. In the embodiment to distinguish protein conformers in a sample comprising a protein having two protein conformations, the method comprises the steps of: (a) reacting the sample with a protein-modifying reagent which reacts differentially with a first protein conformer and a second protein conformer under conditions whereby the reagent forms one or more covalent bond or bonds with the first protein conformer and either does not form a covalent bond with the second protein conformer or forms a covalent bond or bonds with the second protein conformer that is or are different from the covalent bond or bonds formed with the first protein conformer; (b) treating the reacted sample of step (a) with a protein-cleaving reagent under conditions whereby a peptide bond or bonds in the reacted first protein conformer is cleaved to form at least one unique modified peptide and whereby a peptide bond or bonds in the reacted second protein conformer is cleaved to form a unique different peptide from the unique modified peptide of the first protein conformer; and (c) analyzing the treated sample of step (b) to determine the presence of the unique modified peptide of the first protein conformer or to determine the presence of the unique different peptide of the second conformer.

The invention may also include analyzing the treated sample to determine the presence of both the unique modified peptide of the first protein conformer and the unique different peptide of the second protein conformer. The peptides may be identified by various methods such as: mass spectrometric, calorimetric, immunometric, fluorometric, or radiometric detectors with or without prior chromatographic separation.

A further embodiment comprises quantitating the modified peptide formed by the first protein conformer and may also include quantitating the peptide formed by the second protein conformer.

In the embodiment of the invention wherein the protein comprises more than two protein conformations, the method is carried out as above. Using the methods of the invention, chemical markers can be created that are unique to each conformer of the same protein, and the ratio or absolute amounts of each conformer can be determined.

As noted above, in the methods of the invention, the protein is treated with a protein-modifying reagent which reacts differentially with the different protein conformers. In this aspect, the methods of the invention exploit the difference in susceptibility of one or more amino acid residue(s) within different protein conformations, to a protein-modifying reagent by virtue of its changed spatial location and different chemical reactivity. Some amino acid residues are on the surface of the protein in one conformation and hidden in another. An amino acid residue on the surface of one conformer may be susceptible to covalent modification by protein-modifying reagents, while, on another conformer, the same amino acid residue may be buried within the protein and not be susceptible to the same covalent modification. Thus, the invention is effective to detect if only one conformer of a protein is present in the test sample or to distinguish among two or more conformers of a protein that are present in the test sample.

For purposes of this invention, a protein modifying reagent which reacts differentially includes monofunctional reagents (also denoted as monodentate reagents) and bifunctional reagents.

Monofunctional reagents are chemical reagents that possess only one reactive group and result in the modification of a single amino acid or a single class of amino acid, for example, reaction with the free carboxylic acid moiety in ASP or GLU. An example would be acetyl chloride which converts lysine to its acetylated derivative, (but only if that lysine is on the surface of a protein and not involved in a salt bridge to glutamates or aspartates). Another example of a monofunctional reagent is acetic anhydride which results in a variety of differentially modified protein products as described in detail in Example 2, below.

Bifunctional reagents include (a) homobifunctional crosslinking reagents which are chemical reagents with two identical reactive groups (e.g. acyl halide) connected by a linker of varying length; (b) heterobifunctional crosslinking reagents which are chemical reagents with two different reactive groups connected by a linker of varying length and (c) “zero-length” crosslinking reagents which are chemical reagents that lead to internal crosslinks in proteins and result in loss of mass (e.g. loss of H₂ or H₂O). Examples of homobifunctional crosslinking reagents are 1) the amino-specific reagent bis(succinimidyl)suberate (BS³) and 2) ethylene glycobis(succinimidylsuccinate) (EGS). Zero-length cross-linking reagents include oxidants that convert two cysteines to one cysteine (—H₂) and carbodiimides that link aspartate or glutamate to lysine (—H₂O), for example, 1-ethyl-3-(3-dimethylaminopropyl)carbodiimide (EDC). An example of a heterobifunctional crosslinking reagent is N-(alpha-maleimidoacetoxy)-succinimide ester (AMAS) which reacts with both free CYS and amino acids with amino groups (e.g. LYS).

Any of these reagents (monofunctional, homobifunctional or heterobifunctional) may also contain chromophores, fluorophores, radioactive atoms or antibody epitopes to facilitate detection.

Protein-modifying reagents include, but are not limited to, reagents capable of one or more of the following:
Forming a covalent bond with the free α-amino group of the protein conformer.
Forming a covalent bond with the α-amide nitrogen of: Alanine (A), Cysteine (C), Aspartic Acid (D), Glutamic Acid (E), Phenylalanine (F), Glycine (G), Histidine (H), Isoleucine (I), Lysine (K), Leucine (L), Methionine (M), Asparagine (N), Proline (P), Glutamine (Q), Arginine (R), Serine (S), Threonine (T), Valine (V), Tryptophan (W), and/or Tyrosine (Y).
Forming a covalent bond with the hydroxy group of: Serine (S), and/or Threonine (T).
Forming a covalent bond with the phenolic group of: Tyrosine (Y).
Forming a covalent bond with an oxygen of the carboxylate of: Aspartic Acid (D) and/or Glutamic Acid (E).
Forming a covalent bond with the carbon of the carboxylate of: Aspartic Acid (D) and/or Glutamic Acid (E).
Forming a covalent bond with the ω-amide nitrogen of: Asparagine (N) and/or Glutamine (Q).
Forming a covalent bond with the one or more of the guanidino nitrogens of Arginine (R).
Forming a covalent bond with the ε-amine nitrogen of: Lysine (K).
Forming a covalent bond with the aromatic nitrogen of: Histidine (H) or Tryptophan (W).
Forming a covalent bond with the aliphatic carbons of: Alanine (A), Cysteine (C), Aspartic Acid (D), Glutamic Acid (E), Phenylalanine (F), Histidine (H), Isoleucine (I), Lysine (K), Leucine (L), Methionine (M), Asparagine (N), Proline (P), Glutamine (Q), Arginine (R), Serine (S), Threonine (T), Valine (V), Tryptophan (W), and/or Tyrosine (Y).
Forming a covalent bond with the aromatic carbons of: Phenylalanine (F), Histidine (H), Tryptophan (W), and/or Tyrosine (Y).
Forming a covalent bond with the aliphatic α-carbons of: Alanine (A), Cysteine (C), Aspartic Acid (D), Glutamic Acid (E), Phenylalanine (F), Glycine (G), Histidine (H), Isoleucine (I), Lysine (K), Leucine (L), Methionine (M), Asparagine (N), Proline (P), Glutamine (Q), Arginine (R), Serine (S), Threonine (T), Valine (V), Tryptophan (W), and/or Tyrosine (Y).
Forming a covalent bond with the sulfur atoms of: Cysteine (C) and/or Methionine (M).

In addition to the foregoing, the protein-modifying reagent may also be: fluorogenic, chromogenic, biotinylated, immunogenic (reacting with an antibody), covalently bound to a suitable radionucleotide, and/or capable of chelating a rare earth element.

Next, the reacted sample is treated with a protein-cleaving reagent under conditions whereby a peptide bond or bonds in the reacted first protein conformer is cleaved to form at least one unique modified peptide and whereby a peptide bond or bonds in the reacted second protein conformer is cleaved to form a unique different peptide from the unique modified peptide of the first protein conformer. The protein-cleaving reagent can be a chemical agent or a protease.

Chemical agents include, but are not limited to, chemical reagents that cleave amide bonds in proteins such as cyanogen bromide and 2-nitro-5-thiocyanobenzoic acid.

Proteases include: Enzymes capable of cleaving a peptide bond, such as but not limited to proteinase K, trypsin, chymotrypsin; Genetically engineered enzymes that hydrolyze the carboxyl group, in a peptide bond, of the following: Phenylalanine (F), Tryptophan (W), and/or Tyrosine (Y);Genetically engineered enzymes that hydrolyze the carboxyl group, in a peptide bond, of the following: Lysine (K) and/or Arginine (R); Enzymes capable of cleaving a peptide bond that have a strong preference (>10 fold) for cleavage of a specific amino acid at the C or N terminus of the amide bond, such as LYS-C, GLU-C, ARG-C or ASP-N; Enzymes capable of cleaving a peptide bond that have a strong preference (>10 fold) for cleavage of two specific amino acid at the C or N terminus of the amide bond, such as trypsin; Enzymes capable of cleaving a peptide bond that have a strong preference (>10 fold) for cleavage of three specific amino acid at the C or N terminus of the amide bond, such as chymotrypsin; Enzymes capable of cleaving a peptide bond that have a strong preference (>10 fold) for cleavage of four specific amino acid at the C or N terminus of the amide bond; Enzymes capable of cleaving a peptide bond that have a strong preference (>10 fold) for cleavage of five specific amino acid at the C or N terminus of the amide bond; Enzymes capable of cleaving a peptide bond that have a strong preference (>10 fold) for cleavage of six specific amino acid at the C or N terminus of the amide bond.

In cases where the unique peptide(s) contain glycosylation sites, (such as at ASN 181 or ASN 197 in Syrian Hamster PrP), the yield of the unique peptide(s) can be improved by treating the mixture after proteolysis with an enzyme such as PNGase that removes the N-linked oligosaccharides.

Next, the treated sample of step (b) is analyzed to determine the presence of the unique modified peptide of the first protein conformer or determine the presence of the unique different peptide of the second protein conformer. The invention may also include the step of analyzing the treated sample to determine the presence of both unique peptides. The peptides may be identified by various methods such as: mass spectrometric, colorimetric, immunogenic, fluorometric, or radiometric detectors with or without prior chromatographic separation.

Without being limited thereto, analyzing may include using chromatography to separate the peptides and the following methods to identify the peptides: mass spectroscopy; fluorescence; difference in color; difference in ability to bind to streptavidin; difference in the ability to bind to an antibody; difference in the ability to chelate metals.

The methods of the invention are useful for detecting and/or differentiating protein conformers in a sample.

The invention provides a means to detect and/or differentiate prion protein conformers, PrP^{Sc} and PrP^{C}. There is considerable pressure to demonstrate the safety of beef and beef products worldwide, and the invention assay to detect low levels of prions in live animals fills an important need.

The invention can also be used for other prion-based or plaque-forming brain diseases.

Samples for use in the assay include, but are not limited to, tissues and biological fluids such as brain, muscle, blood, tonsil, spleen, and lymphatic tissues and cells in cell culture. Furthermore, in-vitro biochemical samples such as preparations of synaptosomes, liposomes, and endoplasmic reticulum vessicles may be used for this protein conformer assay.

### EXAMPLES

The following examples are intended only to further illustrate the invention and are not intended to limit the scope of the invention which is defined by the claims.

### Example 1

The following example describes differentiation of the two prion conformers, PrP^{C} and PrP^{Sc} in accordance with the methods of the invention, and wherein a bifunctional reagent is used.

Materials and Methods

Reagents

The water-soluble, amino group selective bifunctional cross-linking reagent bis(succinimidyl)suberate (BS³), was purchased from Pierce (Rockford, Ill.). α-Cyano-4-hydroxycinnamic acid (α-CHC) was purchased from Bruker Daltonics (Billerica, Mass.). Achromobacter lysyl endopeptidase (lys C) was obtained from Wako (Osaka, Japan). Trypsin (sequencing grade, modified) was purchased from Promega (Madison, Wis.). An N-Glycosidase F (PNGase F) deglycosylation kit was obtained from New England Biolabs (Beverly, Mass.). All other reagents were from Sigma-Aldrich.

Syrian Hamster PrP27-30

PrP 27-30 was isolated as described (Diringer et al. (1997)) from brains of terminally ill Syrian hamsters infected with the 263K strain of scrapie. Its purity and approximate concentration was assessed by SDS-PAGE and Coomassie blue staining. PrP 27-30 was suspended immediately before use in 1% sarkosyl at an approximate concentration of 0.2 μg/μl by sonication with a 4710 Series probe ultrasonics homogenizer (Cole Parmer, Chicago, Ill.).

Cross-Linking Reactions

PrP 27-30, ˜2-10 μg, was cross-linked in 100 mM phosphate buffer, pH=7.2, at a concentration of 0.067 μg/μl; BS³ was added from a freshly prepared 10 mM stock solution in 5 mM sodium acetate, pH=5, to a final concentration of 1.7 mM, and allowed to react with the protein for 30 minutes at room temperature. The reaction was then terminated by addition of 1M lysine, pH=7.2 to a final concentration of 140 mM and further incubation at room temperature for 15 minutes. Control samples were treated in the same way, except that 5 mM sodium acetate solution, pH=5 was added instead of BS³ solution.

Deglycosylation and Electrophoretic Separation

Cross-linked or control protein was precipitated by centrifugation at 14000 rpm in a table-top centrifuge for 45 minutes; supernatants were carefully aspirated and discarded, and the pellets were rinsed with 200 μl of 85% methanol. Pellets were then denatured and deglycosylated with 3 μl of PNGase solution at 37° C. for 1 hour, according to the manufacturer's instructions. Reaction mixtures were then diluted with an equal volume of reducing Laemmli sample buffer, boiled in 10 minutes and subjected to SDS-PAGE (Laemmli (1970)) using 12 % gels. Protein bands were stained with Coomassie blue.

In-Gel Proteolytic Digestion

For samples analyzed by MALDI-TOF, protein bands were carefully excised with a razor blade, and then reduced, alkylated and digested in-gel with lys C at an approximate mass ratio of 1:10 trypsin or lysC to PrP, according to the procedure of Shevchenko et al. (Shevchenko et al. (1996)) with slight modifications. Briefly, bands were cut to 1 mm³ pieces, placed in an eppendorf tube, washed with water and dehydrated with 200 μl acetonitrile for 15 minutes using mild agitation. Acetonitrile was removed and the gel pieces were dried in vacuo (SpeedVac, Savant, Farmingdale Calif.); a volume of 30 μl of 10 mM DTT in 25 mM NH₄HCO₃ was added and the reduction was carried out at 56° C. for 30 minutes. The solvent was then removed, and after dehydration of gel pieces with acetonitrile as described, replaced with 30 μl of 55 mM iodoacetamide. Alkylation was carried on in the dark at RT for 20 minutes. The solvent was then removed and gel pieces were washed with 25 mM NH₄HCO₃, dehydrated with acetonitrile and rehydrated on ice by addition of 20 μl of 25 mM NH₄HCO₃ containing 15 ng/μl trypsin or lysC. After 40 minutes, 30 μl of 25 mM NH₄HCO₃ were added to cover the gel pieces and samples were incubated overnight at 37° C. Digested samples were briefly centrifuged and the supernatant collected. Gel pieces were then extracted with 20 μl of 25 mM NH₄HCO₃ with sonication for 10 minutes. The solvent was then recovered and replaced with 20 μl of 0.1% trifluoroacetic acid (TFA). The extracts and the digestion solution were pooled and dried in vacuo. Peptides were redissolved in 10 μl of 0.1% TFA, 50% acetonitrile.

For samples analyzed by nanoLC-MS-MS, protein spots were excised from gels then processed in a DigestPro (INTAVIS Bioanalytical Instruments AG, Bergish Gladbach, Germany). Following washing, reduction with DTT, alkylation with iodoacetamide, and in-gel digestion (porcine trypsin, Princeton Separations, Adelphia NJ), the peptides were eluted into a 96 well collection plate with 60 ul of 10% formic acid containing 0.1% trifluoroacetic acid.

MALDI

A 2 μl portion of protein digest was mixed with an equal volume of a saturated solution of α-CHC in acetonitrile/0.1% aqueous TFA (1/2). One μl of the mixture was spotted on a Bruker sample plate, allowed to air-dry and analyzed using a Bruker Autoflex MALDI instrument in reflectron mode. The laser frequency was 5 Hz. About 30 laser shots were averaged.

Nanospray LC/MS/MS

NanoLC-ESI-MS-MS was done with an Applied Biosystems (ABI/MDS Sciex, Toronto, Canada) Model QStar Pulsar equipped with a Proxeon Biosystems (Odense, Denmark) nano-electrospray source. In-gel digest (20μl) was loaded automatically onto a C-18 trap cartridge and chromatographed on a reversed-phase column (Vydac 238EV5.07515, 75 μ×150 mm; Hesperia, Calif.) fitted at the effluent end with a coated spray tip (FS360-50-5-CE, New Objective Inc., Woburn, Mass.). An LC Packings nano-flow LC system (Dionex, Sunnyvale, Calif.) with autosampler, column switching device, loading pump, and nano-flow solvent delivery system was used to elute the column. Elution solvents were: A (0.5% acetic acid in water) and B (80% acetonitrile, 0.5% acetic acid). Samples were eluted at 250 nl/min with the following gradient profile: 2% B at 0 min to 80% B in a 15 min linear gradient; held at 80% B for 5 min then back to 2% B for 10 min. The QStar Pulsar was externally calibrated daily and operated above a resolution of 10,000. The acquisition cycle time of 6s consisted of a single Is MS “survey” scan followed by a 5s MS/MS scan. Ions between m/z 400 to 1,000 of charge states between +2 to +5 having intensities greater than 40 counts in the survey scan were selected for fragmentation. The dynamic exclusion window was set to always exclude previously fragmented masses. Collision energy optimized for charge state and m/z was automatically selected by the Analyst QS software after adjusting parameters to obtain satisfactory fragmentation of GLU fibrinogen peptide (+2), and ACTH (+3 and +4). Nitrogen was used for the collision gas and the pressure in the collision cell ranged from 3×10-6 to 6×10-6 torr.

Data Analysis

LC-MS-MS data was internally calibrated using trypsin autolysis peptides whose identity was confirmed by MS-MS. The MSMS data was submitted to Mascot (Matrix Science) to assist in assignment of unmodified peptides present in the digest. (For these peptides an RMS mass error of 10-15 ppm was observed.) Next, the calibrated TOF-MS survey scans were processed with the “LCMS Reconstruct” tool in the Analyst software. The output is a list of peptide molecular weights calculated by deconvolution of multiple charge states and then identification of the monoisotopic ¹²C species. Lists of peaks present in spectra of monomeric and dimeric cross-linked samples were compiled. Using custom software written in-house, those peaks also present in spectra of control digests of PrP 27-30 were deleted from cross-linked sample peak lists using a 20 ppm mass tolerance. The resulting experimental list of monoisotopic molecular weights was compared to a list of theoretical cross-linked and modified peptides predicted from the sequence of ShaPrP(90-231) using the X-link search tool of GPMaw (http://welcome.to/gpmaw). For MALDI analysis, peaks were externally calibrated using a peptide mix standard (Angiotensin II, Angiotensin I, Substance P, Bombesin, ACTH clip 1-17, ACTH clip 18-39, Somatostatin 28, Bruker); identified major tryptic fragments predicted from the sequence of ShaPrP(90-23 1) were then used to internally calibrate unknown peaks.

Results

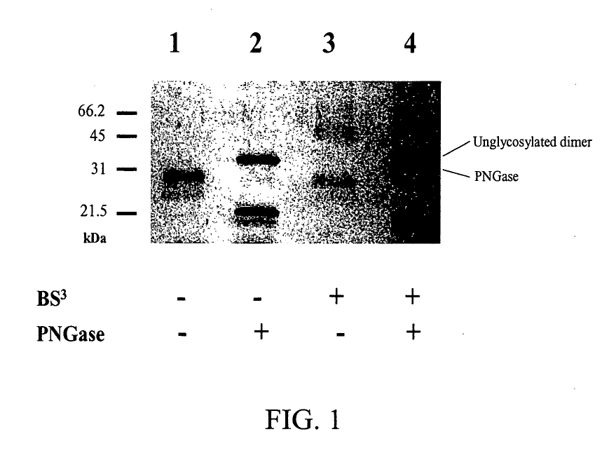

Treatment of PrP 27-30 with BS³ resulted in cross-linked products, documented by the appearance of new bands on polyacrylamide gels corresponding to dimers, trimers and higher order multimers of PrP 27-30 (FIG. 1). Dimers and trimers were also generated at lower concentrations of BS³ (0.17 mM) although oligomer bands were of lower abundance (data not shown). Cross-linking did not interfere with the ability of PNGase F to completely remove carbohydrate chains (FIG. 1). A control experiment in which the order of addition of the cross-linking and quenching reagent was reversed underwent no cross-linking (data not shown). This control ruled out the possibility that the cross-linked species originated artifactually from residual BS³ during sample manipulation subsequent to the cross-linking reaction, i.e. after the native PrP^{Sc} conformation has been lost. Separate control experiments used recombinant Syrian hamster PrP (residues 90-231) at the same reagent and protein concentrations as done with isolated PrP 27-30. Using the same reaction times and temperatures, or changing these variables to potentially increase yields of reacted products resulted in no dimer band as seen by SDS-PAGE in any of the control reactions (data not shown). These control experiments prove that the dimers formed with PrP 27-30, and the crosslinked peptides resulting from these dimers, are formed exclusively from PrP^{Sc} and are not formed from PrP^{C}.

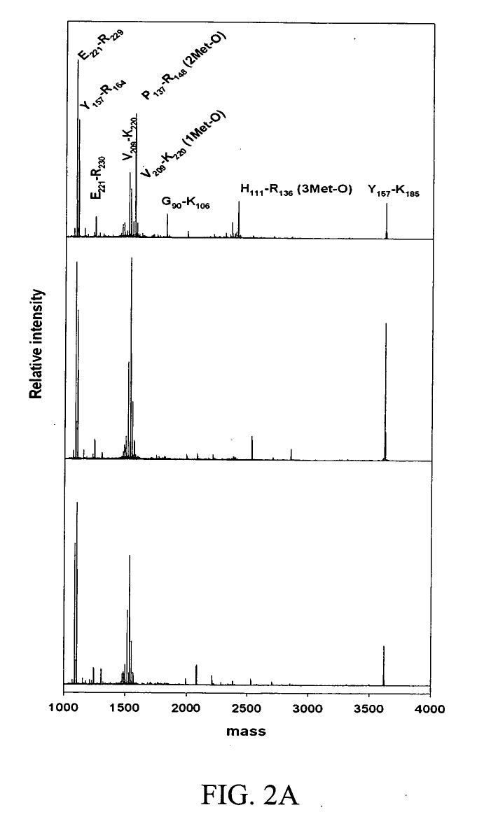

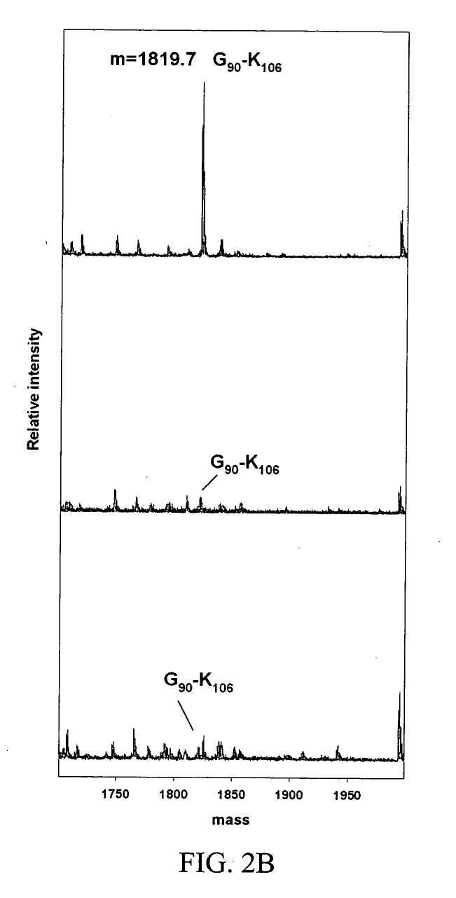

Tryptic digests of PrP 27-30, cross-linked monomer and dimer bands from SDS-PAGE were analyzed by MALDI-TOF (FIG. 2) and nanoLC-ESI-Q-TOF mass spectrometry. Spectra from the cross-linked monomer and dimer bands are very similar to those of control digests of PrP 27-30. Some degree of variability was observed in the relative intensities of some peaks, as a consequence of variable oxidation of peptides containing methionine residues. Also, some variability in cleavage by trypsin at PK positions was observed. Good sequence coverage (88%) of the expected tryptic peptides (Table 1) was observed in all samples and by both techniques. Amino acid residues shown in bold are those which are potential tryptic cleavage sites. The ESI data are summarized in Table 2. RMS mass error for the peptides listed was 12 ppm, and the identity of the indicated sequences was confirmed by MS-MS (data not shown).

TABLE 1
Partial Amino Acid Sequence of Shaprp

```
            86
. . . GGGWGQGGGTHNQWNKPSKPKTNMKHMAGAAAAGAVVGGLGGYMLGSAMSRPMM
HFGNDWEDRYYRENMNRYPNQVYYRPVDQYNNQNNFVHDCVNITIKQHTVTTTTKGENFT
ETDIKIMERVVEQMCTTQYQKESQAYYDGRRS
                                231
```

TABLE 2
Electrospray Ionization (ESI) Data for Example 1

| | Prp 27-30 Control | | | | Prp 27-30 BS3 Dimer | | | Prp 27-30 BS3 Monomer | | |
|---|---|---|---|---|---|---|---|---|---|---|
| | MW | MW | error | relative | MW | error | relative | MW | error | relative |
| peptide | theory | expt'l | (ppm) | int | expt'l | (ppm) | int | expt'l | (ppm) | int |
| 90-106 | 1819.91 | 1819.94 | 15 | 33% | 1819.90 | 6 | 11% | 1819.90 | 7 | 27% |
| 111-136 | 2362.13 | 2362.14 | 3 | 62% | 2362.16 | 9 | 19% | 2362.15 | 7 | 45% |
| 137-148 | 1533.61 | 1533.56 | 33 | 100% | 1533.59 | 15 | 100% | 1533.57 | 26 | 82% |
| 157-164 | 1101.52 | 1101.50 | 22 | 18% | 1101.50 | 19 | 45% | 1101.51 | 18 | 36% |
| 165-185 | 2531.19 | 2531.20 | 3 | 1% | 2531.19 | 1 | 3% | 2531.22 | 14 | 3% |
| 186-194 | 1015.53 | 1015.55 | 20 | 2% | 1015.53 | 3 | 1% | 1015.53 | 3 | 1% |
| 186-204 | 2150.05 | 2150.04 | 5 | 0% | 2150.03 | 10 | 2% | 2150.05 | 2 | 2% |
| 195-204 | 1152.53 | 1152.51 | 18 | 4% | 1152.52 | 6 | 13% | 1152.53 | 4 | 15% |
| 209-220 | 1513.69 | 1513.67 | 17 | 55% | 1513.66 | 18 | 64% | 1513.67 | 17 | 100% |
| 221-229 | 1087.46 | 1087.48 | 21 | 3% | 1087.44 | 17 | 5% | 1087.45 | 5 | 6% |
| 221-230 | 1243.56 | 1243.58 | 21 | 3% | 1243.54 | 14 | 7% | 1243.55 | 11 | 10% |

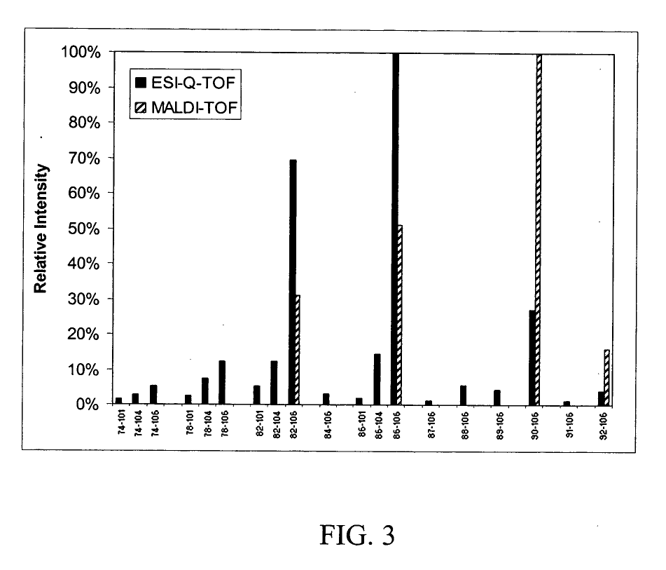

Proteolytic activity of proteinase K used during purification of PrP 27-30 generates a mixture of amino-terminally truncated molecular species. Amino terminal peptides formed by tryptic digestion of these species were identified by MALDI-TOF and nanoLC-ESI-Q-TOF mass spectrometry. Relative abundances of these amino terminal tryptic peptides are shown in FIG. 3. Interpretation is somewhat complicated due to limited tryptic cleavage at 101K/102P and at 104K/105P. Analysis of the ESI data shows three abundant amino termini (GLY 82, GLY 86 and GLY 90), and several other amino termini of low abundance. RMS mass error for the 19 peptides found by ESI was 12 ppm, and the identity of the indicated sequences was confirmed by MS-MS (data not shown). The MALDI results are quite similar. Four abundant amino termini were seen (GLY 82, GLY 86, GLY 90 and GLY 92). ESI data shows the amino terminus at GLY 86 to be most abundant, whereas GLY 90 was found to be most abundant by MALDI.

Comparison of the relative abundances of the amino terminal peptides found in the cross-linked monomer and dimer bands to the amounts seen in PrP 27-30 reveals a very marked, consistent decrease in abundance of all the abundant amino terminal species in the cross-linked samples (FIG. 2B and Table 2). This effect was common to spectra of both trypsin and lysC digests (data not shown). While neither ionization method can yield quantitative data without use of isotopically-labeled standards, these results strongly suggest that amino terminal amino groups extensively react with BS³. The decreased abundances of the amino terminal species in the cross-linked samples is also indicative of exposed locations of the various amino termini allowing facile reaction with the large and ionic crosslinking reagent.

Direct evidence of reaction of the amino termini with BS³ was also observed (Table 3). Four tryptic peptides were found as their suberic acid amides (M+C₈H₁₂O₃), formed by reaction of BS³ with the amino terminus of the peptide followed by hydrolysis of the remaining N-hydroxysuccinimide ester. The modified peptides were assigned to residues 82-106, 86-106, 90-106 and 92-110 which correspond to four of the more abundant amino-terminally truncated PrP 27-30 species (see FIG. 3). RMS mass error for the six measurements (three from the dimer band and three from the monomer band from SDS-PAGE of cross-linked PRP 27-30) was 11 ppm. The identity of the indicated sequences was confirmed by MS-MS (data not shown). The collision-activated dissociation (CAD) spectra of modified peptide 90-106 clearly shows that suberic acid is linked via the amino terminus of the peptide and not via the epsilon-amino group of either LYS 101, LYS 104 or LYS 106. These results demonstrate that GLY 82, GLY 86, GLY 90 and GLY 92 are accessible to the cross-linking reagent and thus must be on the surface of PrP 27-30 fibrils.

TABLE 3
Evidence of Reaction of the Amino Termini with BS³

| | | Prp 27-30 BS3 Dimer | | | Prp 27-30 BS3 Monomer | | |
|---|---|---|---|---|---|---|---|
| | MW | MW | error | relative | MW | error | relative |
| species | theory | expt'l | (ppm) | int | expt'l | (ppm) | int |
| (82 − 106) + C₈H₁₂O₃ | 2752.32 | 2752.31 | 5 | 11% | 2752.37 | 16 | 18% |
| (86 − 106) + C₈H₁₂O₃ | 2333.13 | — | — | 0% | 2333.15 | 11 | 44% |
| (90 − 106) + C₈H₁₂O₃ | 1975.99 | 1975.97 | 9 | 30% | 1975.97 | 9 | 92% |
| (92 − 110) + C₈H₁₂O₃ | 2265.13 | 2265.17 | 15 | 34% | — | — | 0% |
| (90 − 106) + (90 − 106) + C₈H₁₀O₂ | 3777.88 | 3777.83 | 16 | 62% | — | — | 0% |
| (90 − 106) + (86 − 101) + C₈H₁₀O₂ | 3597.70 | 3597.72 | 6 | 12% | — | — | 0% |
| (90 − 106) + (90 − 104) + C₈H₁₀O₂ | 3552.74 | 3552.72 | 4 | 85% | — | — | 0% |
| (90 − 106) + (90 − 101) + C₈H₁₀O₂ | 3240.56 | 3240.56 | 2 | 100% | — | — | 0% |
| (91 − 106) + (92 − 101) + C₈H₁₀O₂ | 2998.45 | 2998.43 | 8 | 59% | — | — | 0% |

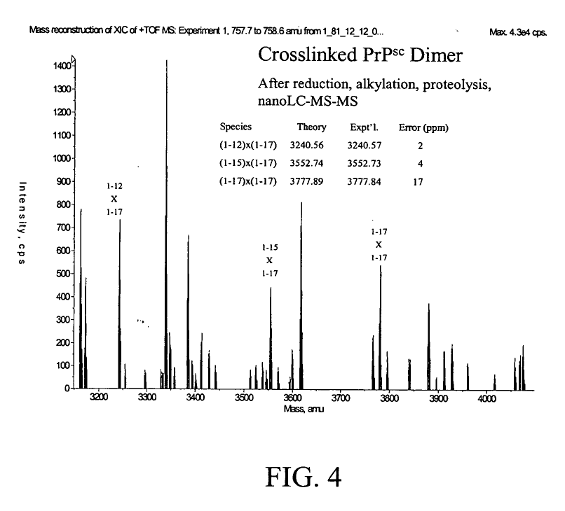

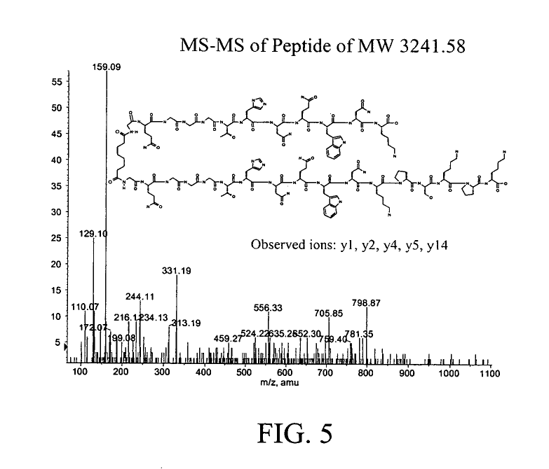

Five intermolecular crosslinked species were also found (Table 3, FIG. 4) in the dimer bands from SDS-PAGE. None were found in the control or monomer bands, as expected. Three of the intermolecular crosslinks arise by crosslinking two PrP 27-30 monomers which both have GLY 90 amino termini; the three species observed differ in the location of their carboxy termini due to limited tryptic cleavage at 101 K/102P and at 104K/105P. The other intermolecular crosslinked species arise from crosslinking PrP 27-30 monomers with amino termini at GLY 90 and GLY 86, or from crosslinking PrP 27-30 monomers with amino termini at GLY 92 and GLY 91, respectively. Due to the difficulty of obtaining good quality MS-MS on small amounts of relatively large molecules (MWs 3,000-4,000), only the most abundant crosslink corresponding to (90-106)+(90-101)+C₈H₁₀O₂ could be confirmed by MS-MS (FIG. 5). Fragmentation observed is fully consistent with the proposed structure.

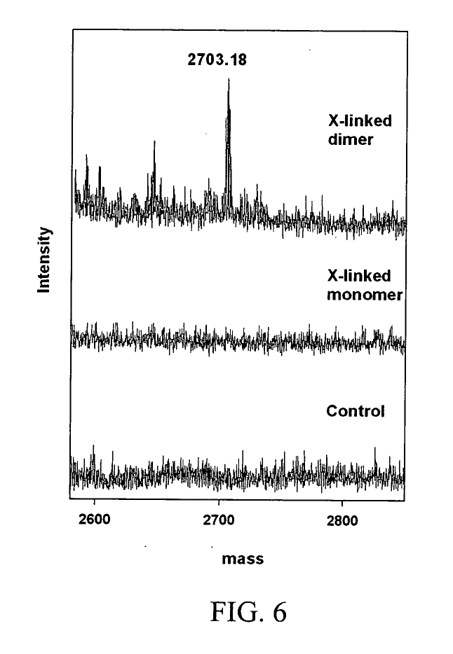

Analysis of the dimer sample by MALDI confirmed the existence of the intermolecular species that is formed by crosslinking two PrP 27-30 monomers which both have GLY 90 amino termini. The evidence was a peak of MW=2703.18 (theoretical mass 2703.23, error=18 ppm), corresponding to an inter-molecular cross-link involving two (90-101) amino terminal peptides, seen in the lysC digest of cross-linked dimeric samples, but not in cross-linked monomers or controls (FIG. 6).

The observation of unmodified amino-terminal peptides (Table 2), and their suberic acid amides (Table 3) in the tryptic digests of crosslinked PrP 27-30 dimers, infers that some of the dimers are crosslinked at sites other than via their amino termini. Comparing the relative abundance of the 90-106 amino-terminal peptide in the PrP 27-30 control digest (33% of base peak intensity) to that found in the crosslinked PrP 27-30 dimer digest (11% of base peak intensity) suggests that as much as 33% of the crosslinked dimers have at least one free amino terminus, and must be crosslinked at sites undetected in this study. The detection of trimers and higher order oligomers in the reaction mixtures substantiates this conclusion.

### Example 2

The following example describes differentiation of the two prion conformers, PrP^{C} and PrP^{Sc} in accordance with the methods of the invention, and wherein a monofunctional reagent (also denoted as a monodentate reagent) is used.

Materials and Methods

Reagents

Trypsin (sequencing grade, modified) was purchased from Promega (Madison, Wis.). Recombinant Syrian hamster prion protein (residues 90-231) was obtained from InPro Biotechnology (South San Francisco, Calif.). All other reagents were from Sigma-Aldrich.

Syrian Hamster PrP27-30

PrP 27-30 was isolated as described (Diringer et al. (1997)) from brains of terminally ill Syrian hamsters infected with the 263K strain of scrapie. Its purity and approximate concentration was assessed by SDS-PAGE and Coomassie blue staining. PrP 27-30 was suspended immediately before use in 1% sarkosyl at an approximate concentration of 0.1 μg/μl by vigorously vortexing to achieve a homogeneous suspension prior to use.

Monofunctional Derivatization

Reactions contained 1 μg of protein in 30 μl of 130 mM phosphate buffer, pH=7.4. Acetic anhydride was added from a freshly prepared stock solution in 20 mM phosphate buffer, pH=7.4, to a final concentration of 7 mM. Reaction was carried out at RT for 15 min and was then quenched by addition of trifluoroacetic acid (TFA) to 1% (v/v) and allowed to incubate at RT for an additional 10 minutes.

Precipitation and Electrophoretic Separation

Protein was precipitated with ice-cold methanol at a final concentration of 85%. The sample was spun in a table-top centrifuge at 14,000 rpm for 20 minutes and the supernatant discarded. The pellet was dissolved in reducing Laemmli buffer, boiled for 10 minutes and subjected to SDS-PAGE (Laemmli (1970)) using 15 % gels. Protein bands were stained with Coomassie blue.

In-Gel Proteolytic Digestion

Protein bands were carefully excised with a razor blade, and then reduced, alkylated and digested in-gel with trypsin at an approximate mass ratio of 1:10 trypsin to PrP, according to the procedure of Shevchenko et al. (Shevchenko et al. (1996)) with slight modifications. Briefly, bands were cut to 1 mm³ pieces, placed in a micro centrifuge tube, washed with water and dehydrated with 200 μl acetonitrile for 15 minutes using mild agitation. Acetonitrile was removed and the gel pieces were dried in vacuo (SpeedVac, Savant, Farmingdale Calif.); a volume of 30 μl of 10 mM DTT in 25 mM NH₄HCO₃ was added and the reduction was carried out at 56° C. for 30 minutes. The solvent was then removed, and after dehydration of gel pieces with acetonitrile as described, replaced with 30 μl of 55 mM iodoacetamide. Alkylation was carried on in the dark at RT for 20 minutes. The solvent was then removed and gel pieces were washed with 25 mM NH₄HCO₃, dehydrated with acetonitrile and rehydrated on ice by addition of 20 μl of 25 mM NH₄HCO₃ containing 10 ng/μl trypsin. After 40 minutes, 30 μl of 25 mM NH₄HCO₃ were added to cover the gel pieces and samples were incubated overnight at 37° C. Digested samples were briefly centrifuged and the supernatant collected. Gel pieces were then extracted with 20 μl of 25 mM NH₄HCO₃ with sonication for 10 minutes. The solvent was then recovered and replaced with 20 μl of 0.1 % trifluoroacetic acid (TFA). The extracts and the digestion solution were pooled and dried in vacuo by centrifugal evaporation (Speed-Vac, Savant).

Nanospray LC/MS/MS

Protein digests were redissolved in 10 μl of 50% acetonitrile containing 1% formic acid, sonicated for 10 min, then diluted with 40 μL of 1% aq. formic acid. NanoLC-ESI-MS-MS was done with an Applied Biosystems (ABI/MDS Sciex, Toronto, Canada) Model QStar Pulsar equipped with a Proxeon Biosystems (Odense, Denmark) nano-electrospray source. In-gel digest (20 μl) was loaded automatically onto a C-18 trap cartridge and chromatographed on a reversed-phase column (Vydac 238EV5.07515, 75 μ×150 mm; Hesperia, Calif.) fitted at the effluent end with a coated spray tip (FS360-50-5-CE, New Objective Inc., Woburn, Mass.). An LC Packings nano-flow LC system (Dionex, Sunnyvale, Calif.) with autosampler, column switching device, loading pump, and nano-flow solvent delivery system was used to elute the column. Elution solvents were: A (0.5% acetic acid in water) and B (80% acetonitrile, 0.5% acetic acid). Samples were eluted at 250 nl/min with the following gradient profile: 2% B at 0 min to 80% B in a 30 min linear gradient; held at 80% B for 5 min then back to 2% B for 10 min. The QStar Pulsar was externally calibrated daily and operated above a resolution of 8,000. The acquisition cycle time of 6s consisted of a single 1s MS “survey” scan followed by a 5s MS/MS scan. Ions between m/z 400 to 1,000 of charge states between +2 to +5 having intensities greater than 40 counts in the survey scan were selected for fragmentation. The dynamic exclusion window was set to always exclude previously fragmented masses. Collision energy optimized for charge state and m/z was automatically selected by the Analyst QS software after adjusting parameters to obtain satisfactory fragmentation of GLU fibrinogen peptide (+2), and ACTH (+3 and +4). Nitrogen was used for the collision gas and the pressure in the collision cell ranged from 3×10-6 to 6×10-6 torr.

Data Analysis

To assist in assignment of unmodified peptides present in the digest the MSMS data was submitted to Mascot (Matrix Science). For these peptides an RMS mass error of 20-30 ppm was observed. Next, the TOF-MS survey scans were processed with the “LCMS Reconstruct” tool in the Analyst software. The output is a list of peptide molecular weights calculated by deconvolution of multiple charge states and then identification of the monoisotopic ¹²C species. The data was then searched manually to find peptides modified by acetic anhydride. MSMS of relevant peaks was also interpreted manually.

Results

Treatment of PrP 27-30 or recombinant PrP 90-231 (rPrP90-23 1) with acetic anhydride resulted in differentially modified protein products. Tryptic digests of PrP 27-30, or recombinant PrP 90-231 after reaction with acetic anhydride were analyzed by nanoLC-ESI-Q-TOF mass spectrometry and were found to contain modified peptides, some of which were unique to each protein conformer. The ESI data are summarized in Table 4. Four classes of peptides were found. Two examples of each are described.

Class 1. Peptides unique to PrP^{Sc}: Examples shown are 1-17 having reacted once with acetic anhydride and 1-17 unmodified. MSMS of 1-17 conclusively shows the site of modification to be at Gly1, the amino terminal amino acid.

Class 2. Peptides unique to PrP^{C}: Examples shown are 97-115 having reacted once with acetic anhydride, and 106-119 having reacted once with acetic anhydride and found as the methionine sulfoxide (a common oxidation product of methionine often observed in proteins purified by SDS-PAGE). MSMS data of these peptides conclusively shows that the epsilon-amino group of the internal lysine residues (Lys105 for 97-115, and Lys115 for 106-119) are the sites of acetylation.

Class 3. Modified peptides found in both PrP^{C} and PrP^{Sc} but in significantly different amounts. Examples shown are 1-17 having reacted twice with acetic anhydride and 1-17 having reacted three times with acetic anhydride. MSMS data of 1-17 having reacted two times with acetic anhydride clearly shows the sites of modification at Gly1 (the amino terminus) and Lys12, but not at Lys15 or Lys17. MSMS data of 1-17 having reacted three times with acetic anhydride clearly shows the sites of modification at Gly 1 (the amino terminus), Lys 12 and Lys 15, but not at Lys 17.

Class 4. Peptides found in both PrP^{C} and PrP^{Sc} but in essentially the same amount. Examples shown are 48-59 and 68-75. Both these peptides have carboxy-terminal arginines, are preceded by arginine, and contain no residues likely react with acetic anhydride.

RMS mass error for the peptides listed in Table 4 is 25 ppm which is consistent with the range of errors observed for the entire set of unmodified peptides (see above). The identity of all the peptides listed in Table 4 (with the exception of unmodified 1 -17 which co-eluted with more abundant peaks) was confirmed by MS-MS (data not shown).

TABLE 4
Electrospray Ionization (ESI) Data for Example 2

| | | | Peptides from recombinant PrP | | | Peptides from PrP 27-30 | | |
|---|---|---|---|---|---|---|---|---|
| | | MW | MW | error | rel | MW | error | rel |
| peptide | modifications | theory | expt'l | (ppm) | intensity | expt'l | (ppm) | intensity |
| 1-17 | none | 1819.91 | — | — | 0 | 1819.84 | 37 | 11 |
| 1-17 | Ac_1 | 1861.92 | — | — | 0 | 1861.84 | 44 | 4 |
| 1-17 | Ac_2 | 1903.93 | 1903.90 | 13 | 3 | 1903.86 | 38 | 33 |
| 1-17 | Ac_3 | 1945.94 | 1945.91 | 14 | 14 | 1945.96 | 11 | 1 |
| 97-115 | Ac_1 | 2192.06 | 2192.04 | 8 | 4 | — | — | 0 |
| 106-119 | 1 oxygen and | 1739.80 | 1739.77 | 19 | 26 | — | — | 0 |
| | Ac_1 | | | | | | | |
| 48-59 | 2 oxygens | 1565.60 | 1565.57 | 22 | 100 | 1565.54 | 38 | 100 |
| 68-75 | none | 1101.52 | 1101.50 | 21 | 38 | 1101.48 | 41 | 33 |

Modification “Ac_x” refers to the indicated peptide having reacted x times with acetic anhydride.

It is understood that the foregoing detailed description is given merely by way of illustration and that modification and variations may be made within, without departing from the spirit and scope of the invention. All publications and patents cited herein are hereby incorporated by reference in their entirety.

### REFERENCES

AGUZZI, A., and Polymenidou, M., (2004) Cell 116:313-327.
COME, J. H. et al., (1993) Proc. Natl. Acad. Sci. 90:5959-5936.
DIRINGER, H. et al., (1997) Intervirology 40:238-246.
LAEMMLI, U. K., (1970) Nature 227:680-685.
MCKINLEY, M. P. et al., (1991) J. Virology 65(3): 1340-1351.
PEPYS, M. B., (1996) Amyloidosis. In Weatherhall, D. J, Ledingham, J. G. G. and Warell, D. A. (Eds), The Oxford Textbook of Medicine, 3rd edition, Vol 2, Oxford University Press, Oxford, UK, 1512-1524.
PRUSINER, S. B., (1998) Proc. Natl. Acad. Sci. 95:13363-13383.
PRUSINER, S. B., (1991) Science 252:1515-1522.
PRUSINER, S. B., (1982) Science 216(9):136-144.
REQUENA, J. R. et al., (2001) Proc. Natl. Acad. Sci. 98(13):7170-7175.
RIEK, R. et al., (1996) Nature 382:180-182.
SHEVCHENKO et al., (1996) Anal. Chem. 68(5):850-858.
SOTO, C., and Castilla, J., (2004) Nat. Med. 7 Suppl. S63-S67.
STAHL, N. et al., (1993) Biochemistry 32:1991-2002.

We Claim:

1. A method for detecting, distinguishing or quantitating a protein conformer in a sample comprising a protein having a plurality of protein conformers, wherein each conformer is characterized by a unique protein conformation, comprising:
(a) reacting the sample with a protein-modifying reagent which reacts differentially with each of the plurality of protein conformers under conditions whereby the reagent forms one or more covalent bonds with a first one of the plurality of protein conformers to form a first unique entity, and whereby a second entity corresponding to each additional protein conformer results either because the reagent does not form one or more covalent bonds with a second one of the plurality of protein conformers or because the reagent forms one or more covalent bonds with a second one of the plurality of protein conformers wherein the one or more covalent bond is different from the one or more covalent bonds formed with the first protein conformer;
(b) treating the reacted sample of step (a) with a protein-cleaving reagent under conditions whereby one or more peptide bonds in the first entity is cleaved to form at least one unique modified peptide and whereby one or more peptide bonds in the reacted second entity is cleaved to form a unique different peptide which differs from the unique modified peptide of the first entity; and
(c) analyzing the treated sample of step (b) to determine the presence of the unique modified peptide of the first entity or to determine the presence of the unique different peptide of the second entity.

2. The method of claim 1, wherein the analyzing in step (c) of the treated sample of step (b) is to both determine the presence of the unique modified peptide of the first entity and to determine the presence of the unique different peptide of the second entity.

3. The method of claim 1, wherein the plurality of protein conformers comprises more than two protein conformers.

4. The method of claim 1, wherein at least one of said protein conformers is quantitated.

5. The method of claim 1, wherein said protein-modifying reagent is a monofunctional reagent.

6. The method of claim 5, wherein said monofunctional reagent is selected from the group consisting of acetyl chloride and acetic anhydride.

7. The method of claim 1, where said protein-modifying reagent is a bifunctional reagent.

8. The method of claim 7, where said protein-modifying reagent is a homobifunctional reagent.

9. The method of claim 7, where said protein-modifying reagent is a heterobifunctional reagent.

10. The method of claim 7, wherein said bifunctional reagent is selected from the group consisting of bis(succinimidyl)suberate, ethylene glycobis(succinimidylsuccinate), 1-ethyl-3-(3-dimethylaminopropyl)carbodiimide, and N-(alpha-maleimidoacetoxy)-succinimide ester.

11. The method of claim 1, wherein said protein-modifying reagent reacts with one or more amino acid residues on one of said plurality of protein conformers.

12. The method of claim 1, wherein said protein-modifying reagent is fluorogenic, chromogenic, biotinylated, immunogenic, covalently bound to a suitable radionucleotide, and/or capable of chelating a rare earth element.

13. The method of claim 1, wherein said protein-cleaving reagent in step (b) is a chemical reagent.

14. The method of claim 1, wherein said protein-cleaving reagent in step (b) is a protease.

15. The method of claim 1, wherein said analyzing in step (c) employs a detector selected from the group consisting of mass spectrometric, colorimetric, immunometric, fluorometric, and radiometric systems.

16. The method of claim 15, wherein said analyzing in step (c) further comprises subjecting said sample to chromatographic separation prior to employing said detector.

17. The method of claim 1, wherein said protein is a protein that forms amyloid deposits.

18. The method of claim 1, wherein said protein is prion protein and said one or more protein conformer is selected from the group consisting of normal prion protein (PrP^{C}), the infectious isoform of normal prion protein (PrP^{Sc}), and mixtures thereof.

19. The method of claim 1, wherein said sample is a biological tissue or fluid.

20. The method of claim 19, wherein said biological tissue or fluid is selected from the group consisting of brain, muscle, blood, tonsil, spleen, and lymph.
